(19) United States
(12) Patent Application Publication
(10) Pub. No.: US 2024/0412876 A1
(43) Pub. Date: Dec. 12, 2024

(54) INVALID FEATURE MANAGEMENT FOR COMPOSITE HEALTH INDEX

(71) Applicant: Cardiac Pacemakers, Inc., St. Paul, MN (US)

(72) Inventors: Viktoria A. Averina, Shoreview, MN (US); Pramodsingh Hirasingh Thakur, Woodbury, MN (US); Mojgan Goftari, Shoreview, MN (US)

(21) Appl. No.: 18/732,278

(22) Filed: Jun. 3, 2024

Related U.S. Application Data
(60) Provisional application No. 63/472,548, filed on Jun. 12, 2023.

(51) Int. Cl. G16H 50/30; A61B 5/00; G16H 40/67

(52) U.S. Cl. CPC G16H 50/30; A61B 5/7221; G16H 40/67

(57) ABSTRACT

Systems and methods are disclosed to determine a composite health index for the patient as a function of a plurality of features, including determining validity of a first feature of the plurality of features and adjusting the function used to determine the composite health index in response to a determination that the first feature is not valid.

### CLAIM OF PRIORITY

This application claims the benefit of U.S. Provisional Patent Application Ser. No. 63/472,548, filed on Jun. 12, 2023, the benefit of priority of which is claimed hereby, and which is incorporated by reference herein in its entirety.

### TECHNICAL FIELD

This document relates generally to medical devices and more particularly to managing invalid features for a composite health index.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine a composite health index for the patient as a function of a plurality of features, including determining validity of a first feature of the plurality of features and adjusting the function used to determine the composite health index in response to a determination that the first feature is not valid.

An example of subject matter (e.g., a medical device system) may comprise a signal receiver circuit configured to receive physiologic information of a patient, the physiologic information including a plurality of features and an assessment circuit configured to determine a composite health index for the patient as a function of at least two of the plurality of features, including to determine validity of a first feature of the at least two of the plurality of features; and in response to a determination that the first feature of the at least two of the plurality of features is not valid, adjust the function used to determine the composite health index.

In an example, to adjust the function used to determine the composite health index comprises substituting a second feature of the plurality of features for the first feature in response to the determination that the first feature is not valid, wherein the second feature is correlative to the first feature.

In an example, which may be combined with any one or more of the previous examples, the assessment circuit is configured to determine a correlation between the first feature and the second feature, and to determine that the second feature is correlative to the first feature if a determined correlation between the first feature and the second feature over a first time period preceding the determination that the first feature is not valid exceeds a threshold correlation.

In an example, which may be combined with any one or more of the previous examples, the first time period preceding the determination that the first feature is not valid is between 3 days and 31 days.

In an example, which may be combined with any one or more of the previous examples, the assessment circuit is configured to select the second feature from two or more additional features of the plurality of features separate from the first feature, including to determine a correlation between the first feature and the two or more additional features over a first time period preceding the determination that the first feature is not valid and select the second feature based on the determined correlations.

In an example, which may be combined with any one or more of the previous examples, the function to determine the composite health index of the patient does not include the two or more additional features when the first feature is valid.

In an example, which may be combined with any one or more of the previous examples, to substitute the second feature of the plurality of features for the first feature in response to the determination that the first feature is not valid comprises to assign a weight for the second feature in the function corresponding to a weight of the first feature before the determination that the first feature is not valid.

In an example, which may be combined with any one or more of the previous examples, to adjust the function used to determine the composite health index comprises to use a last valid value of the first feature for a second time period including a first number of days after the determination that the first feature is not valid, and thereafter, to substitute the second feature for the first feature.

In an example, which may be combined with any one or more of the previous examples, to adjust the function used to determine the composite health index comprises to remove the first feature from the function and to adjust a weight of a valid one of the at least two of the plurality of features in the function in response to the determination that the first feature is not valid.

In an example, which may be combined with any one or more of the previous examples, to adjust the weight of the valid one of the at least two of the plurality of features comprises to maintain relative weight of remaining valid features of the at least two of the plurality of features in the function.

In an example, which may be combined with any one or more of the previous examples, to adjust the weight of the valid one of the at least two of the plurality of features comprises to increase the weight over a first specified number of days from a previous weight from before the determination that the first feature is not valid to a target weight higher than the previous weight.

In an example, which may be combined with any one or more of the previous examples, to adjust the weight of the valid one of the at least two of the plurality of features comprises to keep the weight constant for a second specified number of days after the determination that the first feature is not valid before increasing the weight.

In an example, which may be combined with any one or more of the previous examples, to adjust the function used to determine the composite health index comprises to use a representation of the first feature based on a third time period in response to the determination that the first feature is not valid.

In an example, which may be combined with any one or more of the previous examples, to adjust the function used to determine the composite health index comprises to decrease a weight of the representation of the first feature over time and to increase a weight of remaining valid features of the at least two of the plurality of features commensurate with the decreased weight of the representation of the first feature over time.

In an example, which may be combined with any one or more of the previous examples, in response to the determination that the first feature is not valid, the assessment circuit is configured determine one of a physiologic or a non-physiologic reason for the determination and to adjust the function used to determine the composite health index includes using a first adjustment in response to a determined physiologic reason and using a second adjustment in response to a determined non-physiologic reason, wherein the first adjustment is different than the second adjustment.

In an example, which may be combined with any one or more of the previous examples, the composite health index includes a composite heart failure index.

In an example, which may be combined with any one or more of the previous examples, a method includes receiving, using a signal receiver circuit, physiologic information of a patient, the physiologic information including a plurality of features, determining, using an assessment circuit, a composite health index for the patient as a function of at least two of the plurality of features, the determining comprising determining validity of a first feature of the at least two of the plurality of features and adjusting the function used to determine the composite health index in response to determining that the first feature of the at least two of the plurality of features is not valid.

In an example, which may be combined with any one or more of the previous examples, adjusting the function used to determine the composite health index comprises substituting a second feature of the plurality of features for the first feature in response to determining that the first feature is not valid, wherein the second feature is correlative to the first feature.

In an example, which may be combined with any one or more of the previous examples, the method includes determining, using the assessment circuit, a correlation between the first feature and the second feature and determining that the second feature is correlative to the first feature if a determined correlation between the first feature and the second feature over a first time period preceding determining that the first feature is not valid exceeds a threshold correlation.

In an example, which may be combined with any one or more of the previous examples, the first time period preceding determining that the first feature is not valid is between 3 days and 31 days.

In an example, which may be combined with any one or more of the previous examples, the method includes selecting, using the assessment circuit, the second feature from two or more additional features of the plurality of features separate from the first feature, including determining a correlation between the first feature and the two or more additional features over a first time period preceding determining that the first feature is not valid, and selecting the second feature based on the determined correlations.

In an example, which may be combined with any one or more of the previous examples, determining the composite health index as the function of at least two of the plurality of features does not include using the two or more additional features when the first feature is valid.

In an example, which may be combined with any one or more of the previous examples, substituting the second feature of the plurality of features for the first feature in response to determining that the first feature is not valid comprises assigning a weight for the second feature in the function corresponding to a weight of the first feature before determining that the first feature is not valid.

In an example, which may be combined with any one or more of the previous examples, adjusting the function used to determine the composite health index comprises using a last valid value of the first feature for a second time period including a first number of days after determining that the first feature is not valid, and thereafter, substituting the second feature for the first feature.

In an example, which may be combined with any one or more of the previous examples, adjusting the function used to determine the composite health index comprises removing the first feature from the function and adjusting a weight of a valid one of the at least two of the plurality of features in the function in response to determining that the first feature is not valid.

In an example, which may be combined with any one or more of the previous examples, adjusting the weight of the valid one of the at least two of the plurality of features comprises maintaining relative weight of remaining valid features of the at least two of the plurality of features in the function.

In an example, which may be combined with any one or more of the previous examples, adjusting the weight of the valid one of the at least two of the plurality of features comprises increasing the weight over a first specified number of days from a previous weight from before determining that the first feature is not valid to a target weight higher than the previous weight.

In an example, which may be combined with any one or more of the previous examples, adjusting the weight of the valid one of the at least two of the plurality of features comprises keeping the weight constant for a second specified number of days after determining that the first feature is not valid before increasing the weight.

In an example, which may be combined with any one or more of the previous examples, adjusting the function used to determine the composite health index comprises using a representation of the first feature based on a third time period in response to determining that the first feature is not valid.

In an example, which may be combined with any one or more of the previous examples, adjusting the function used to determine the composite health index comprises decreasing a weight of the representation of the first feature over time and increasing a weight of remaining valid features of the at least two of the plurality of features commensurate with the decreased weight of the representation of the first feature over time.

In an example, which may be combined with any one or more of the previous examples, the method includes in response to determining that the first feature of the at least two of the plurality of features is not valid, determining one of a physiologic or a non-physiologic reason for the determination, where adjusting the function used to determine the composite health index comprises using a first adjustment in response to a determined physiologic reason and using a second adjustment in response to a determined non-physiologic reason, wherein the first adjustment is different than the second adjustment.

In an example, which may be combined with any one or more of the previous examples, the composite health index includes a composite heart failure index.

In an example, a system or apparatus may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example medical device system.

FIG. 2 illustrates an example patient management system.

FIG. 3 illustrates an example method for managing an invalid feature in a composite health index.

FIG. 4 illustrates an example implantable medical device (IMD) electrically coupled to a heart.

FIG. 5 illustrates a block diagram of an example machine upon which any one or more of the techniques discussed herein may perform.

### DETAILED DESCRIPTION

Ambulatory medical devices can include, or be configured to receive physiologic information from, one or more sensors located within, on, or proximate to a body of a patient. Physiologic information of the patient can include, among other things, respiration information (e.g., a respiratory rate, a respiration volume (tidal volume)), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

One or more composite health indexes can be determined, in certain examples, as a function of different physiologic information of the patient or various combinations thereof. In certain examples, a composite health index can be a device-based index, without input of clinical information about the patient, such as clinician diagnosis or determination of risk, patient history, patient age, comorbidities, prior hospitalization, type of implanted device, etc. In other examples, the composite health index can be a combination of a device-based and clinical-based mortality risk index, including or taking into account clinical information about the patient, such as clinician diagnosis or determination of risk, patient history, patient age, comorbidities, prior hospitalization, type of implanted device, etc. In an example, separate determinations can be made for different combinations of clinical information.

One example of a composite health index is a HeartLogic™ index, a HeartLogic™ in-alert time, or one or more other composite measurements or measures thereof. The HeartLogic™ index is a composite measurement from multiple ambulatory sensors, including S1 and S3 heart sounds, thoracic impedance, activity information, respiration information, and nighttime heart rate (nHR), indicative of a heart failure status, a risk of heart failure event, or a worsening of the heart failure status or risk of heart failure event in the patient over time. The HeartLogic™ in-alert time is a measure of time that the HeartLogic™ index is above an alert threshold.

In certain examples, the HeartLogic™ index can be determined using different combinations or weightings of physiologic information, including one or more of S1 and S3 heart sounds, thoracic impedance, activity information, rapid shallow breathing index (RSBI), respiratory rate, and nighttime heart rate (nHR). In certain examples, the different combinations or weightings of the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.).

For example, if the risk stratifier is low, or below a first threshold, the HeartLogic™ index can be determined using a first combination of physiologic information. If the risk stratifier is high, or above a second threshold, the HeartLogic™ index can be determined using the first combination of physiologic information and a second combination of physiologic information, including additional information than included in the first combination. If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

Implantable and ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Historically, heart sounds were assessed by humans and thus only the audible portion of the vibration was used. Devices can now assess a broader spectrum of vibrations, such as can include a full spectrum of cardiac vibrations which can include both audible and subaudible components, thus the term “sound” in this application refers to full spectrum of vibrations. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure of respiratory frequency relative tidal volume of the patient. The nHR is a measure of heart rate (HR) of the patient at night, either in relation to sensing patient sleep or using a preset or selectable time of day corresponding to patient sleep.

Physiologic values or features, as described herein, can include one or more different measures of rate, amplitude, energy, etc., of different physiologic information over one or more time periods, such as representative daily values, etc. For example, heart sound values can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily values can be determined representative of an average daily value for the patient, either corresponding to a waking time or a 24-hour period, etc. Respiration values can include, among other things, a mean or median respiration rate, binned values of rates, and a representative value of specific rate bins, etc. Heart rate values can include an average nighttime heart rate, a minimum nighttime heart rate, etc.

The activity information can include an activity measurement of the patient, such as detected using an accelerometer, a posture sensor, a step counter, or one or more other activity sensors associated with an ambulatory medical device. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In certain examples, one or more sensor values or features used to determine a composite health index, such as the HeartLogic™ index, can be or become invalid. In certain examples, a feature can include a sensor value or can be determined using one or more sensor values. A sensor value can be determined as invalid based on one or more conditions, such as if the value is lost, is above or below one or more thresholds, changes more than a threshold amount between successive values or over a period of time, if the value contradicts one or more other sensed values, etc. In other examples, a sensor value can be determined as invalid if it cannot be determined with a high degree of confidence that the sensor value is valid, such as if the value is outside of one or more valid or expected ranges, if the value contradicts one or more other sensed values having a higher determined confidence, etc.

For example, a sensor value may be determined as not valid if the sensor value is lost (e.g. if a memory buffer is exceeded), if a rate of change of the sensor value exceeds a threshold (e.g., if the sensor value rises or falls at a rate exceeding a threshold), if the sensor value goes outside specified bounds (e.g., if the sensor exceeds a configurable minimum or maximum bound), or if the sensor value contradicts one or more other sensor values (e.g., if the sensor value is different from a sensor value from a redundant sensor, the sensor value is different from another sensor value correlative with the value measured by the sensor, the sensor value contradicts another sensor value that typically exhibits a correlation with the sensor value, such as based on a history of sensor values for the patient, etc.). In other examples, a sensor value may be determined as not valid if another sensor value is outside specified bounds (e.g., S3 may not be able to be assessed when heart rate is above a specified rate or above a specified variability), if its trending is disabled (e.g., if RRT trending is turned off via programmer), if there are fewer than the needed number of raw data samples for its computation (e.g., if the daily value of RR needs at least 60% of valid breaths measured during that day), if sensing is suspended due to another event or activity (e.g. during an MRI scan), if a sensing feature is undergoing calibration or initialization (e.g. heart sound engine uses 7 days of data collection to initialize its parameters). Sensors may be more likely to take measurements that are not valid when the sensors are one or more of operating on low power to conserve energy, such as may be due to a low power sensor being less robust, operating at a low measurement frequency to conserve energy, such as may be due to the longer time length between measurements allowing for the potential of a larger change in sensor values between measurements, which may make it difficult to determine if the changes are the result of normal operating conditions, or positioned in an insertable cardiac monitor (ICM) device as opposed to a transvenous device, which may result in the sensors being farther from the source of the signals to be measured.

If one or more sensor values or features used in the determination of a composite health index are determined as not valid, the function used to determine the composite health index can be adjusted to improve a sensitivity or specificity of the composite health index, such as in contrast to maintaining the existing function using the invalid sensor value or feature or setting the sensor value or feature to zero. For example, maintaining the existing function including the invalid value or removing the invalid value from the function may reduce a clinical value of the composite health index or trigger one or more device modes or operations that reduce or limit the effectiveness of the medical device.

The present inventors have recognized, among other things, systems and methods to manage an invalid feature used to determine a composite health index, such as to reduce a negative impact of the invalid feature. In an example, a system can determine a validity of one or more features in a function used to determine the composite health index and, in response to determining that one or more of the features are invalid, adjust the function to remove, replace, or accommodate the one or more invalid features.

For example, the system can remove the invalid feature and adjust a weight of one or more of the remaining features, such as to avoid or reduce a step change or discontinuity in an output of the composite health index. In certain examples, a weight of one or more remaining valid features can be increased until an output of the composite health index matches a value of the output before the invalid feature was determined to be invalid. In an example, relative weights of the remaining valid features can be preserved when increasing the weights of features in the function. In certain examples, a weight of one or more remaining valid features can be adjusted based upon a time that the invalid feature was determined to be invalid or a number of invalid features in the function.

In other examples, the system can substitute one or more features into the function in place of the one or more invalid features. In certain examples, the weight, effective weight, value, or effective value of the substituted feature can be determined based on the weight or value of the invalid feature before the invalid feature was determined to be invalid. The substitute feature can be selected, in certain examples, based on a determined correlation between one or more previous values of the invalid feature before the invalid feature was determined to be invalid and one or more other features. For example, the system (e.g., an assessment circuit, etc.) can determine a correlation between two or more features distinct from the function used to determine the composite health index and the invalid feature over a range of values before the invalid feature was determined to be invalid and select or recommend one or more features having a highest determined correlation or a determined correlation above one or more thresholds as one or more substitute features.

In other examples, the system can determine a representative value of the invalid feature based upon one or more values or a range of values of the invalid feature before the invalid feature was determined to be invalid and replace the invalid feature with the determined representative value. For example, the system can substitute the last valid value of the invalid feature from before the invalid feature was determined to be invalid as the determined representative value. In an example, the system can determine the representative value based on one or more trends of one or more values or a range of values of the invalid feature before the invalid feature was determined to be invalid (e.g., a value predicted by a curve fit to historical data). In an example, a weight of the invalid feature can be decayed out the longer the feature is invalid, such as may result in the invalid feature having no effect on the function after a specified length of time. In certain examples, a weight of one or more remaining valid features or one or more substitute features or representative values can be increased commensurate to the decay of the weight of the invalid feature.

In certain examples, the techniques described above or herein can be used in various combinations or permutations. For example, combinations or permutations of techniques described above or herein can be selected to reduce or minimize discontinuities in the output of the composite health index (e.g., matching effective weights or decaying a weight of the invalid feature, etc.).

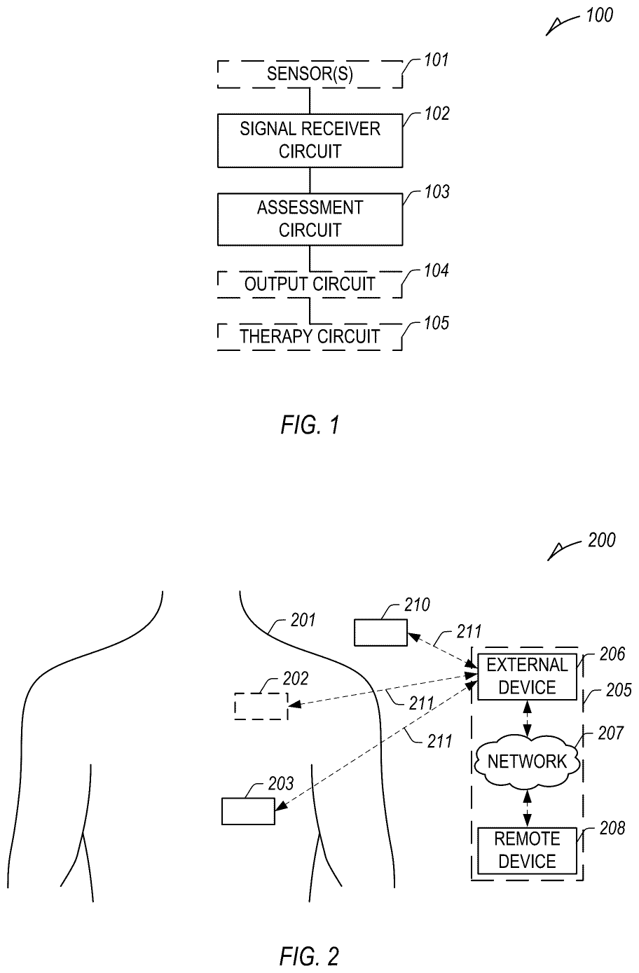

FIG. 1 illustrates an example system 100 (e.g., a medical device system). In an example, one or more aspects of the example system 100 can be a component of, or communicatively coupled to, a medical device, such as an implantable medical device (IMD), an insertable cardiac monitor, an ambulatory medical device (AMD), etc. The system 100 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 100 can include a single medical device or a plurality of medical devices implanted in a patient's body or otherwise positioned on or about the patient to monitor patient physiologic information of the patient using information from one or more sensors, such as a sensor 101. In an example, the sensor 101 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 100 can include a signal receiver circuit 102 and an assessment circuit 103. The signal receiver circuit 102 can be configured to receive physiologic information of a patient (or group of patients) from the sensor 101. The assessment circuit 103 can be configured to receive information from the signal receiver circuit 102, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 103 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 100 can include an output circuit 104 configured to provide an output to a user, or to cause an output to be provided to a user, such as through an output, a display, or one or more other user interface, the output including a score, a trend, an alert, or other indication. In other examples, the output circuit 104 can be configured to provide an output to another circuit, machine, or process, such as a therapy circuit 105 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 105 can include one or more of a stimulation control circuit, a cardiac stimulation circuit, a neural stimulation circuit, a dosage determination or control circuit, etc. In other examples, the therapy circuit 105 can be controlled by the assessment circuit 103, or one or more other circuits, etc. In certain examples, the assessment circuit 103 can include the output circuit 104 or can be configured to determine the output to be provided by the output circuit 104, while the output circuit 104 can provide the signals that cause the user interface to provide the output to the user based on the output determined by the assessment circuit 103.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 2 illustrates an example patient management system 200 and portions of an environment in which the patient management system 200 may operate. The patient management system 200 can perform a range of activities, including remote patient monitoring and diagnosis of a disease condition. Such activities can be performed proximal to a patient 201, such as in a patient home or office, through a centralized server, such as in a hospital, clinic, or physician office, or through a remote workstation, such as a secure wireless mobile computing device.

The patient management system 200 can include one or more medical devices, an external system 205, and a communication link 211 providing for communication between the one or more ambulatory medical devices and the external system 205. The one or more medical devices can include an ambulatory medical device (AMD), such as an implantable medical device (IMD) 202, a wearable medical device 203, or one or more other implantable, leadless, subcutaneous, external, wearable, or medical devices configured to monitor, sense, or detect information from, determine physiologic information about, or provide one or more therapies to treat various conditions of the patient 201, such as one or more cardiac or non-cardiac conditions (e.g., dehydration, sleep disordered breathing, etc.).

In an example, the implantable medical device 202 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 201. In another example, the implantable medical device 202 can include a monitor implanted, for example, subcutaneously in the chest of patient 201, the implantable medical device 202 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 202 can include an assessment circuit configured to detect or determine specific physiologic information of the patient 201, or to determine one or more conditions or provide information or an alert to a user, such as the patient 201 (e.g., a patient), a clinician, or one or more other caregivers or processes, such as described herein. The implantable medical device 202 can alternatively or additionally be configured as a therapeutic device configured to treat one or more medical conditions of the patient 201. The therapy can be delivered to the patient 201 via the lead system and associated electrodes or using one or more other delivery mechanisms. The therapy can include delivery of one or more drugs to the patient 201, such as using the implantable medical device 202 or one or more of the other ambulatory medical devices, etc. In some examples, therapy can include CRT for rectifying dyssynchrony and improving cardiac function in heart failure patients. In other examples, the implantable medical device 202 can include a drug delivery system, such as a drug infusion pump to deliver drugs to the patient for managing arrhythmias or complications from arrhythmias, hypertension, hypotension, or one or more other physiologic conditions. In other examples, the implantable medical device 202 can include one or more electrodes configured to stimulate the nervous system of the patient or to provide stimulation to the muscles of the patient airway, etc.

The wearable medical device 203 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 205 can include a dedicated hardware/software system, such as a programmer, a remote server-based patient management system, or alternatively a system defined predominantly by software running on a standard personal computer. The external system 205 can manage the patient 201 through the implantable medical device 202 or one or more other ambulatory medical devices connected to the external system 205 via a communication link 211. In other examples, the implantable medical device 202 can be connected to the wearable medical device 203, or the wearable medical device 203 can be connected to the external system 205, via the communication link 211. This can include, for example, programming the implantable medical device 202 to perform one or more of acquiring physiologic data, performing at least one self-diagnostic test (such as for a device operational status), analyzing the physiologic data, or optionally delivering or adjusting a therapy for the patient 201. Additionally, the external system 205 can send information to, or receive information from, the implantable medical device 202 or the wearable medical device 203 via the communication link 211. Examples of the information can include real-time or stored physiologic data from the patient 201, diagnostic data, such as detection of patient hydration status, hospitalizations, responses to therapies delivered to the patient 201, or device operational status of the implantable medical device 202 or the wearable medical device 203 (e.g., battery status, lead impedance, etc.). The communication link 211 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 205 can include an external device 206 in proximity of the one or more ambulatory medical devices, and a remote device 208 in a location relatively distant from the one or more ambulatory medical devices, in communication with the external device 206 via a communication network 207. Examples of the external device 206 can include a medical device programmer. The remote device 208 can be configured to evaluate collected patient or patient information and provide alert notifications, among other possible functions. In an example, the remote device 208 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 208 can receive data from multiple patients. The data can be collected by the one or more ambulatory medical devices, among other data acquisition sensors or devices associated with the patient 201. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 208 may additionally include one or more locally configured clients or remote clients securely connected over the communication network 207 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 208, including the server and the interconnected clients, may also execute a follow-up scheme by sending follow-up requests to the one or more ambulatory medical devices, or by sending a message or other communication to the patient 201 (e.g., the patient), clinician or authorized third party as a compliance notification.

The communication network 207 can provide wired or wireless interconnectivity. In an example, the communication network 207 can be based on the Transmission Control Protocol/Internet Protocol (TCP/IP) network communication specification, although other types or combinations of networking implementations are possible. Similarly, other network topologies and arrangements are possible.

One or more of the external device 206 or the remote device 208 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 206 or the remote device 208 can include a respective display unit for displaying the physiologic or functional signals, or alerts, alarms, emergency calls, or other forms of warnings to signal the detection of arrhythmias. In some examples, the external system 205 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 205 can be implemented using hardware, software, firmware, or combinations thereof. Portions of the one or more ambulatory medical devices or the external system 205 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 210 can be configured to send information to or receive information from one or more of the ambulatory medical devices or the external system 205 using the communication link 211. In an example, the one or more ambulatory medical devices, the external device 206, or the remote device 208 can be configured to control one or more parameters of the therapy device 210. The external system 205 can allow for programming the one or more ambulatory medical devices and can receives information about one or more signals acquired by the one or more ambulatory medical devices, such as can be received via a communication link 211. The external system 205 can include a local external implantable medical device programmer. The external system 205 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

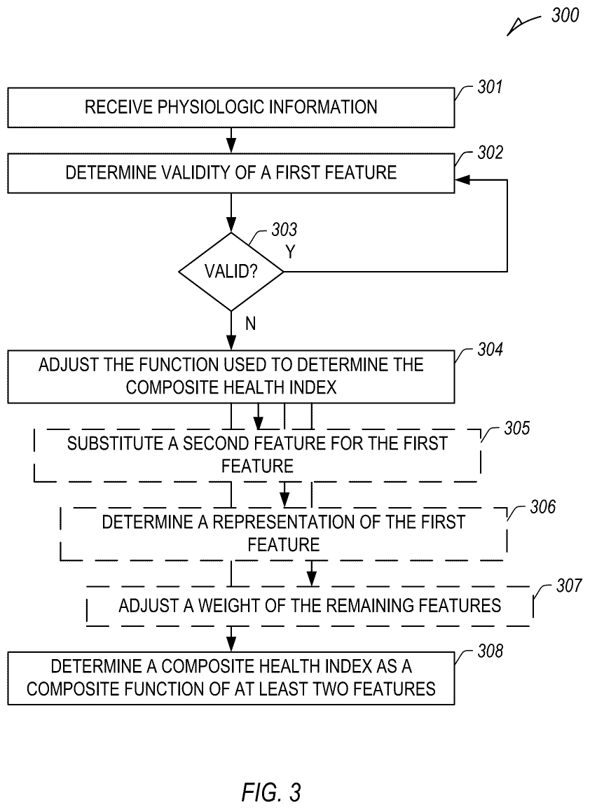

FIG. 3 illustrates an example method 300 for replacing, adjusting, or otherwise managing one or more invalid features in a composite function having multiple features or parameters for determining a composite health metric, such as described herein.

At step 301, physiologic information of a patient can be received, such as using a signal receiver circuit. The physiologic information of the patient can include at least one of respiration information (e.g., respiratory rate, tidal volume, RSBI, etc.), cardiac electrical information (e.g., heart rate, impedance, etc.), impedance information, cardiac acceleration information (e.g., heart sounds, etc.), mechanical acceleration information (e.g., activity information, heart sounds, etc.), mechanical position information (e.g., patient posture, sleep incline, etc.).

At step 302, a validity of a first feature can be determined, such as using the assessment circuit. In certain examples, a feature can include a sensor value or can be determined using one or more sensor values. A sensor value can be determined as invalid based on one or more conditions, such as if the value is lost, changes more than a threshold amount between successive values or over a period of time, is above or below one or more thresholds, if the value contradicts one or more other sensed values, etc. In other examples, a sensor value can be determined as invalid if another sensor value is outside specified bounds, if its trending is disabled, if there are fewer than the needed number of raw data samples for its computation, if sensing is suspended due to another event or activity, or if a sensing feature is undergoing calibration or initialization. In still other examples, a sensor value can be determined as invalid if it cannot be determined with a high degree of confidence that the sensor value is valid, such as if the value is outside of one or more valid or expected ranges, if the value contradicts one or more other sensed values having a higher determined confidence, if the feature provides a step increase in one or more measures, such as a composite health index (e.g., the HeartLogic index, etc.), greater than a static threshold or one or more thresholds determined based on a patient history of previous changes, etc. For example, if the value of a composite health index changes greater than a threshold, the feature or combination of features causing the change can be identified, such as in relation to the remaining features and previous values, and the validity of such features can be determined. In certain examples, validity of the individual features are not determined until the value of the composite health index changes greater than the threshold. In other examples, validity of the individual features can be determined at regular intervals. Although described in the remaining steps with respect to the composite health index, such method is applicable to other measures, composite measures, etc.

In some examples, the step 302 may optionally include determining the validity of one or more additional features (e.g., one or more additional features not used to determine the composite health index, etc.), such as to compare relative changes between different features. In certain examples, validity of the first feature can be determined with respect to one or more other features. In certain examples, validity of at least two of the plurality of features can be determined.

At step 303, if the first feature is determined to be valid, the method can include returning to step 302 to re-check validity. The validity of the first feature can be determined recurrently, such as can include recurrently at a periodic interval. Additionally, validity of one or more other features can be determined. In an example, the periodic interval can be one or more of five minutes, one hour, six hours, twelve hours, or one day.

If the first feature is determined to not be valid at step 303, the function used to determine the composite health index (e.g., which up to this point included the first feature, etc.) can be adjusted at step 304, such as using an assessment circuit. Adjusting the function used to determine the composite health index can include, among other things, adjusting the function to remove, replace, or accommodate the one or more invalid features.

For example, at step 305, a second feature can optionally be substituted for the first feature, such as using an assessment circuit. In certain examples, the weight, effective weight, value, or effective value of the substituted feature can be determined based on the weight or value of the invalid feature before the invalid feature was determined to be invalid. The substitute feature can be selected, in certain examples, based on a determined correlation between one or more previous values of the invalid feature before the invalid feature was determined to be invalid and one or more other features. For example, a correlation can be determined between two or more features distinct from the function used to determine the composite health index and the invalid feature over a range of values before the invalid feature was determined to be invalid. One or more features having a highest determined correlation or a determined correlation above one or more thresholds can be selected or recommended as one or more substitute features.

The two or more features may be distinct from the function used to determine the composite health index in the sense that the function used to determine the composite health index may not include the two or more additional features when the first feature is valid. For example, when the first feature is valid, the two or more additional features may not have an impact on the value of the composite health index.

In an example, at step 305, a second feature may be substituted for the first feature if the second feature is determined to have a correlation to the first feature that exceeds a threshold correlation. A correlation between the first feature and the second feature may be determined using a mathematical technique (e.g., Pearson correlation coefficient, sum of squares, etc.). A threshold correlation may be variable or static. In an example, the threshold may be a static, preconfigured threshold that is not adjusted during operation. The preconfigured threshold value may be determined to help ensure the accuracy of the composite health index. In an example, the threshold may be a variable threshold, such as may include a relative threshold or a threshold that may be adjusted (e.g., adjusted by the assessment circuit) based upon one or more determinations. For example, the threshold correlation may be increased if the composite health index has been relatively stable for a period of time or decreased if the composite health index has been variable. In an example, the threshold correlation may be reduced or increased based upon the weight of the invalid feature. For example, the threshold may be reduced if the feature has a high weight because it may be important to have a component representing the feature in the composite health index. In an example, the threshold may be reduced if the feature has a low weight, because it may be less critical that the representative feature accurately mimic the invalid feature.

The correlation between the first and second feature may be determined over a first time period preceding the determination that the first feature is not valid. The time period may be selected to help enable the selection of a second feature that accurately represents the first feature. In an example, the first time period may be between 3 and 31 days. Data less than 3 days old may not be desirable, as the first feature may have been less accurate in recent time, such as may be due to the feature becoming invalid. This may result in the data of the first feature not accurately representing the metric the first feature is intended to track, which can result in selectin a second feature that does not accurately represent the metric that the first feature is intended to track. Data over 31 days old may have limited or diminishing relevance, such as may be due to one or more of changes in patient condition, changes in the measurement of various features, etc. In an example, the correlation between the first and second feature may be predetermined based on previously collected clinical study data where correlation is assessed during specific time periods (e.g., a clinical study conducted 30 days before heart failure events).

At step 306, a representation of the first feature can optionally be determined, such as using an assessment circuit. The representation of the first feature may include a representative value of the invalid feature based upon one or more values or a range of values of the invalid feature before the invalid feature was determined to be invalid. The first feature may be replaced by the determined representative value. For example, the last valid value of the invalid feature from before the invalid feature was determined to be invalid can be substituted as the determined representative value. In an example, the representative value can be determined based on one or more trends of one or more values or a range of values of the invalid feature before the invalid feature was determined to be invalid (e.g., a value predicted by a curve fit to historical data). In an example, a weight of the invalid feature can be decayed out the longer the feature is invalid, such as may result in the invalid feature having no effect on the function after a specified length of time. In certain examples, a weight of one or more remaining valid features or one or more substitute features or representative values can be increased commensurate to the decay of the weight of the invalid feature.

In an example, the representation of the first feature that is substituted for the invalid first feature may be determined based on values of the first feature from a third time period. The third time period can include between 3 days to 10 days preceding the determination that the first feature is not valid. Using more than a single historical value of the first feature may help provide a better approximation of the first feature (e.g., linear averaging, weighted averaging favoring more recent measurements, etc.). Avoiding the use of the most recent data may help to reduce the effect of the first feature near the time it is determined to be not valid, such as may include while it is becoming invalid.

At step 307, a weight of the remaining features can be adjusted, such as using an assessment circuit. For example, the invalid feature can be removed and a weight of one or more of the remaining features can be adjusted, such as to avoid or reduce a step change or discontinuity in an output of the composite health index. In certain examples, a weight of one or more remaining valid features can be increased until an output of the composite health index matches a value of the output before the invalid feature was determined to be invalid. In an example, relative weights (i.e., the ratio of the weights) of the remaining valid features can be preserved when increasing the weights of features in the function. In certain examples, a weight of one or more remaining valid features can be adjusted based upon a time that the invalid feature was determined to be invalid or a number of invalid features in the function.

The weight of the one or more remaining valid features may be increased over a first specified number of days, such as may include 3 days, 7 days, or 10 days. For example, the weights may be increased from a previous weight from before the determination that the first feature is not valid to a target weight, such as may be higher than the previous weight. The first specified number of days may be included to help prevent a step discontinuity in the composite health metric. In an example, the weight of the one or more remaining valid features may be held constant for a second specified number of days after the determination that the first feature is not valid before increasing. The second specified number of days may include 1 day, 3 days, or 5 days. The second specified number of days may be included to allow the invalid feature time to become valid again or increase the time that the composite health index is determined using the original features.

In an example, the function may be adjusted differently based upon the determined reason for invalidity of the first feature. In addition to determining the validity of the first feature at 302, the reason for invalidity may be determined. The reason for invalidity may be a physiologic reason (e.g., a physical condition of the patient is making the first feature in valid, for example, the S3 heart sound does not fully occur if the heart rate is too high or variable causing the pacing to interrupt or prevents diastole) or a non-physiologic reason (e.g., a condition within the hardware, such as a buffer overflow or an initialization in progress). The determination of whether the feature is invalid for physiologic or non-physiologic reasons may be used in one or more steps of the method 300. For example, if the feature is invalid due to a physiologic reason, the weight of remaining features may be increased, in addition to substituting a second feature for the first feature or determining a representation of the first feature. This increase in weight of the remaining features may make the function more sensitive (e.g., a change in one of the other features will be magnified when the first feature is invalid for a physiologic reason). This increase in sensitivity may be beneficial because invalidity due to physiologic reasons may be a sign of worsening health in and of itself (e.g., the condition being invalid for physiologic reasons is a sign that the composite health index should be indicating a worsening health metric). If the feature is invalid for a non-physiologic reason (e.g., indicating that it is a hardware/software failure as opposed to a physiologic condition of the patient), the function may be adjusted as discussed above (e.g., without increasing the sensitivity of remaining features), or not adjusted at all. In an example, when the first feature is invalid for physiologic reasons, the substituted feature may be selected differently (e.g., a feature indicating the abnormality of the heart rate may be substituted for the invalid heart sound). In an example, when the first feature is invalid for physiologic reasons, an additional feature may be added to the function, in addition to using a substitute feature or representation for the invalid feature. This additional feature may cause the composite health index to indicate a worsening condition even if the remaining features are unchanged.

In certain examples, the techniques of any one or more of steps 304-307 can be used in various combinations or permutations. In certain examples, any one or more of steps 304-307 can apply to more than one invalid feature.

At step 308, the composite health index can be determined as a function of at least two features, such as using an assessment circuit. The composite health index can be determined using the adjusted function. In an example where the first feature is determined to be valid, the composite health index may be determined at 308 before the validity of the first feature is re-checked at 302. (e.g., if all features in the composite health index are determined to be valid, the composite health index can be computed with the unadjusted function). The at least two features can include functions of the physiologic information received by the signal receiver circuit. For example, a feature corresponding to a breathing rate could be calculated with a high feature value corresponding to a healthy breathing rate and low feature value corresponding to an unhealthy breathing rate. A high breathing rate (e.g., 30 breaths per minute) can be assigned a value of 100 and a low breathing rate (e.g., 12 breaths per minute) can be assigned a value of 0. The function mapping between the high breathing rate and low breathing rate can be assigned one or more of linearly, logarithmically, etc. The composite health index can include a function of the different features, such as one or more of a weighted combination (e.g., average, product, summation, etc.) of two or more features (e.g., each feature having a corresponding weight). The combination can include a linear combination or one or more non-linear or other combinations.

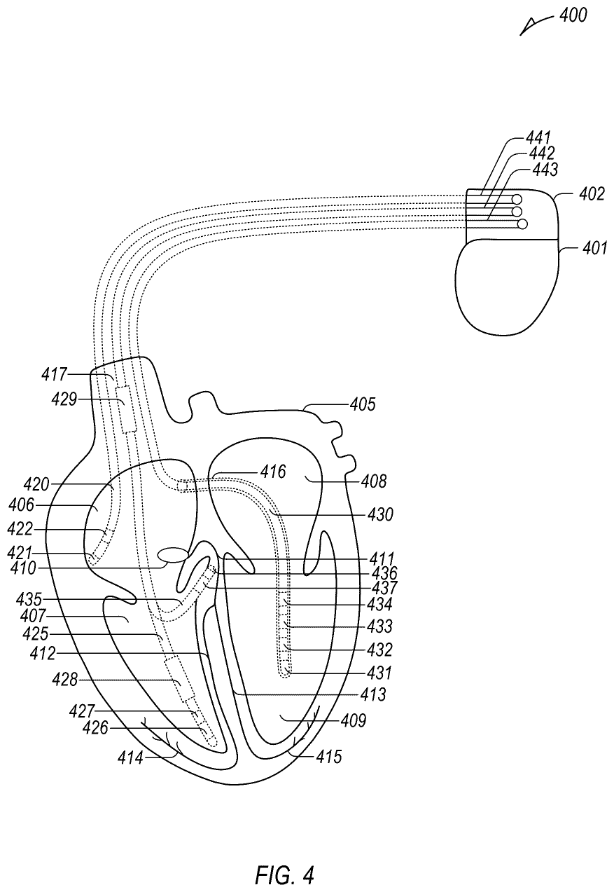

FIG. 4 illustrates an implantable medical device (IMD) 400 electrically coupled to a heart 405, such as through one or more leads coupled to the IMD 400 through one or more lead ports, such as first, second, or third lead ports 441, 442, 443 in a header 402 of the IMD 400. In an example, the IMD 400 can include an antenna, such as in the header 402, configured to enable communication with an external system and one or more electronic circuits (e.g., an assessment circuit, etc.) in a hermetically sealed housing (CAN) 401. The IMD 400 illustrates an example medical device (or a medical device system) as described herein.

The IMD 400 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 405, such as in one or more of the atria or ventricles. Separate from, or in addition to, the one or more electrodes or other sensors of the leads, the IMD 400 can include one or more electrodes or other sensors (e.g., a pressure sensor, an accelerometer, a gyroscope, a microphone, etc.) powered by a power source in the IMD 400. The one or more electrodes or other sensors of the leads, the IMD 400, or a combination thereof, can be configured detect physiologic information from, or provide one or more therapies or stimulation to, the patient.

The IMD 400 can include one or more electronic circuits configured to sense one or more physiologic signals, such as an electrogram or a signal representing mechanical function of the heart 405. In certain examples, the CAN 401 may function as an electrode such as for sensing or pulse delivery. For example, an electrode from one or more of the leads may be used together with the CAN 401 such as for unipolar sensing of an electrogram or for delivering one or more pacing pulses. A defibrillation electrode (e.g., the first defibrillation coil electrode 428, the second defibrillation coil electrode 429, etc.) may be used together with the CAN 401 to deliver one or more cardioversion/defibrillation pulses.

In an example, the IMD 400 can sense impedance such as between electrodes located on one or more of the leads or the CAN 401. The IMD 400 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 400 can be configured to inject current between an electrode on one or more of the first, second, third, or fourth leads 420, 425, 430, 435 and the CAN 401, and to sense the resultant voltage between the same or different electrodes and the CAN 401.

The example lead configurations in FIG. 4 include first, second, and third leads 420, 425, 430 in traditional lead placements in the right atrium (RA) 406, right ventricle (RV) 407, and in a coronary vein 416 (e.g., the coronary sinus) over the left atrium (LA) 408 and left ventricle (LV) 409, respectively, and a fourth lead 435 positioned in the RV 407 near the His bundle 411, between the AV node 410 and the right and left bundle branches 412, 413 and Purkinje fibers 414, 415. Each lead can be configured to position one or more electrodes or other sensors at various locations in or near the heart 405 to detect physiologic information or provide one or more therapies or stimulation.

The first lead 420, positioned in the RA 406, includes a first tip electrode 421 located at or near the distal end of the first lead 420 and a first ring electrode 422 located near the first tip electrode 421. The second lead 425 (dashed), positioned in the RV 407, includes a second tip electrode 426 located at or near the distal end of the second lead 425 and a second ring electrode 427 located near the second tip electrode 426. The third lead 430, positioned in the coronary vein 416 over the LV 409, includes a third tip electrode 431 located at or near the distal end of the third lead 430, a third ring electrode 432 located near the third tip electrode 431, and two additional electrodes 433, 434. The fourth lead 435, positioned in the RV 407 near the His bundle 411, includes a fourth tip electrode 436 located at or near the distal end of the fourth lead 435 and a fourth ring electrode 437 located near the fourth tip electrode 436. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 425 includes a first defibrillation coil electrode 428 located near the distal end of the second lead 425 in the RV 407 and a second defibrillation coil electrode 429 located a distance from the distal end of the second lead 425, such as for placement in or near the superior vena cava (SVC) 417.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 411.

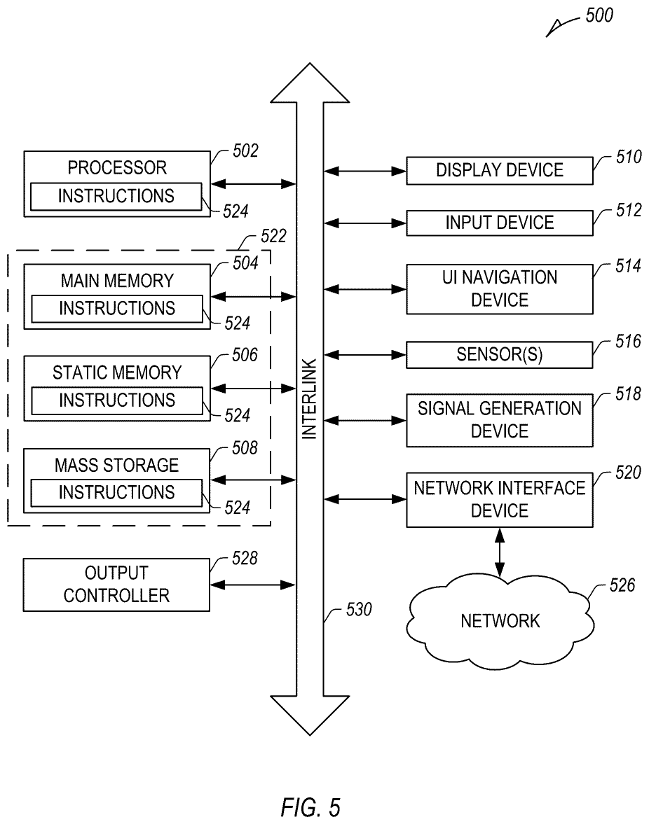

FIG. 5 illustrates a block diagram of an example machine 500 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 500. Circuitry (e.g., processing circuitry, an assessment circuit, etc.) is a collection of circuits implemented in tangible entities of the machine 500 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 500 follow.

In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 500 (e.g., computer system) may include a hardware processor 502 (e.g., a central processing unit (CPU), a graphics processing unit (GPU), a hardware processor core, or any combination thereof), a main memory 504, a static memory 506 (e.g., memory or storage for firmware, microcode, a basic-input-output (BIOS), unified extensible firmware interface (UEFI), etc.), and mass storage 508 (e.g., hard drive, tape drive, flash storage, or other block devices) some or all of which may communicate with each other via an interlink 530 (e.g., bus). The machine 500 may further include a display unit 510, an input device 512 (e.g., a keyboard), and a user interface (UI) navigation device 514 (e.g., a mouse). In an example, the display unit 510, input device 512, and UI navigation device 514 may be a touch screen display. The machine 500 may additionally include a signal generation device 518 (e.g., a speaker), a network interface device 520, and one or more sensors 516, such as a global positioning system (GPS) sensor, compass, accelerometer, or one or more other sensors. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 502, the main memory 504, the static memory 506, or the mass storage 508 may be, or include, a machine-readable medium 522 on which is stored one or more sets of data structures or instructions 524 (e.g., software) embodying or utilized by any one or more of the techniques or functions described herein. The instructions 524 may also reside, completely or at least partially, within any of registers of the hardware processor 502, the main memory 504, the static memory 506, or the mass storage 508 during execution thereof by the machine 500. In an example, one or any combination of the hardware processor 502, the main memory 504, the static memory 506, or the mass storage 508 may constitute the machine-readable medium 522. While the machine-readable medium 522 is illustrated as a single medium, the term “machine-readable medium” may include a single medium or multiple media (e.g., a centralized or distributed database, and/or associated caches and servers) configured to store the one or more instructions 524.

The term “machine-readable medium” may include any medium that is capable of storing, encoding, or carrying instructions for execution by the machine 500 and that cause the machine 500 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may be further transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 500, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient, the physiologic information including a plurality of features; and
an assessment circuit configured to determine a composite health index for the patient as a function of at least two of the plurality of features, including to:
determine validity of a first feature of the at least two of the plurality of features; and
in response to a determination that the first feature of the at least two of the plurality of features is not valid, adjust the function used to determine the composite health index.

2. The medical device system of claim 1, wherein to adjust the function used to determine the composite health index comprises substituting a second feature of the plurality of features for the first feature in response to the determination that the first feature is not valid, wherein the second feature is correlative to the first feature.

3. The medical device system of claim 2, wherein the assessment circuit is configured to determine a correlation between the first feature and the second feature, and to determine that the second feature is correlative to the first feature if a determined correlation between the first feature and the second feature over a first time period preceding the determination that the first feature is not valid exceeds a threshold correlation.

4. The medical device system of claim 3, wherein the first time period preceding the determination that the first feature is not valid is between 3 days and 31 days.

5. The medical device system of claim 2, wherein the assessment circuit is configured to select the second feature from two or more additional features of the plurality of features separate from the first feature, including to:
determine a correlation between the first feature and the two or more additional features over a first time period preceding the determination that the first feature is not valid; and
select the second feature based on the determined correlations.

6. The medical device system of claim 5, wherein the function to determine the composite health index of the patient does not include the two or more additional features when the first feature is valid.

7. The medical device system of claim 2, wherein to substitute the second feature of the plurality of features for the first feature in response to the determination that the first feature is not valid comprises to assign a weight for the second feature in the function corresponding to a weight of the first feature before the determination that the first feature is not valid.

8. The medical device system of claim 2, wherein to adjust the function used to determine the composite health index comprises to use a last valid value of the first feature for a second time period including a first number of days after the determination that the first feature is not valid, and thereafter, to substitute the second feature for the first feature.

9. The medical device system of claim 1, wherein to adjust the function used to determine the composite health index comprises to remove the first feature from the function and to adjust a weight of a valid one of the at least two of the plurality of features in the function in response to the determination that the first feature is not valid.

10. The medical device system of claim 9, wherein to adjust the weight of the valid one of the at least two of the plurality of features comprises to maintain relative weight of remaining valid features of the at least two of the plurality of features in the function.

11. The medical device system of claim 9, wherein to adjust the weight of the valid one of the at least two of the plurality of features comprises to increase the weight over a first specified number of days from a previous weight from before the determination that the first feature is not valid to a target weight higher than the previous weight.

12. The medical device system of claim 11, wherein to adjust the weight of the valid one of the at least two of the plurality of features comprises to keep the weight constant for a second specified number of days after the determination that the first feature is not valid before increasing the weight.

13. The medical device system of claim 1, wherein to adjust the function used to determine the composite health index comprises to use a representation of the first feature based on a third time period in response to the determination that the first feature is not valid.

14. The medical device system of claim 13, wherein to adjust the function used to determine the composite health index comprises to decrease a weight of the representation of the first feature over time and to increase a weight of remaining valid features of the at least two of the plurality of features commensurate with the decreased weight of the representation of the first feature over time.

15. The medical device system of claim 1, wherein, in response to the determination that the first feature is not valid, the assessment circuit is configured determine one of a physiologic or a non-physiologic reason for the determination, and
wherein to adjust the function used to determine the composite health index includes using a first adjustment in response to a determined physiologic reason and using a second adjustment in response to a determined non-physiologic reason, wherein the first adjustment is different than the second adjustment.

16. The medical device system of claim 1, wherein the composite health index includes a composite heart failure index.

17. A method, comprising:
receiving, using a signal receiver circuit, physiologic information of a patient, the physiologic information including a plurality of features; and
determining, using an assessment circuit, a composite health index for the patient as a function of at least two of the plurality of features, including:
determining validity of a first feature of the at least two of the plurality of features; and
adjusting the function used to determine the composite health index in response to determining that the first feature of the at least two of the plurality of features is not valid.

18. The method of claim 17, wherein adjusting the function used to determine the composite health index comprises substituting a second feature of the plurality of features for the first feature in response to determining that the first feature is not valid, wherein the second feature is correlative to the first feature.

19. The method of claim 17, wherein adjusting the function used to determine the composite health index comprises removing the first feature from the function and adjusting a weight of a valid one of the at least two of the plurality of features in the function in response to determining that the first feature is not valid.

20. The method of claim 17, wherein adjusting the function used to determine the composite health index comprises using a representation of the first feature based on a third time period in response to determining that the first feature is not valid.
